(19) United States
(12) Patent Application Publication
(10) Pub. No.: US 2025/0049977 A1
(43) Pub. Date: Feb. 13, 2025

(54) Dynamically Adjusting Scent for Scent Dispensation

(71) Applicant: Pura Scents, Inc., Pleasant Grove, UT (US)

(72) Inventors: Trevor Davis, American Fork, UT (US); Sam Biggs, Salt Lake City, UT (US); Dane Thomas, Lehi, UT (US); Andrew Wallin, Millcreek, UT (US)

(21) Appl. No.: 18/800,809

(22) Filed: Aug. 12, 2024

Related U.S. Application Data
(60) Provisional application No. 63/519,155, filed on Aug. 11, 2023.

(51) Int. Cl. A61L 9/12; H04L 67/125

(52) U.S. Cl. CPC A61L 9/122; H04L 67/125; A61L 2209/111; A61L 2209/133; A61L 2209/16

(57) ABSTRACT

A scent dispensing device may perform dynamic scent diffusion by identifying a scent type in a scent container of the fragrance management apparatus, determining a room dimension where the fragrance management apparatus is installed, determining a removal rate of a remaining scent in the scent container, determining a concentration level based on settings of the scent container, calculating a ppm output of the fragrance management apparatus for the scent type based on the room dimensions, the removal rate, and the concentration level, and causing the fragrance management apparatus to maintain a fragrance level based on the ppm output.

### BACKGROUND

The present disclosure relates to dynamically adjusting scent for scent dispensation.

Existing solutions for dispensing scents within a room include liquid scented oil diffusers, wax or cork disks infused with scented oils, or nebulizers. The scented oil can be dispersed throughout the room by heating elements and/or fans. Additionally, some existing solutions for dispensing scents allow users to manually select scent intensity so that a user can adjust scent dispensation to their preferences. However, manual selections of scent dispensation often result in scents being too strong or too weak for specific rooms and users are unable to optimize that scent solution using manual selections.

### SUMMARY

In some aspects, the techniques described herein relate to a fragrance management apparatus, including: a non-volatile memory; and a controller of a fragrance management apparatus, the controller configured to execute instructions stored on the non-volatile memory to perform operations including: identifying a scent type in a scent container of the fragrance management apparatus; determining a room dimension where the fragrance management apparatus is installed; determining a removal rate of a remaining scent in the scent container; determining a concentration level based on settings of the scent container; calculating a ppm output of the fragrance management apparatus for the scent type based on the room dimension, the removal rate, and the concentration level; and causing the fragrance management apparatus to maintain a fragrance level based on the ppm output.

In some aspects, the techniques described herein relate to a fragrance management apparatus, wherein the scent type is a scent solution that emits a scent when heated and the scent container is a scent vial that contains the scent solution.

In some aspects, the techniques described herein relate to a fragrance management apparatus, wherein the room dimension is determined based on an identity of the room that categorizes the room dimensions.

In some aspects, the techniques described herein relate to a fragrance management apparatus, wherein categorizing the room dimensions further includes: determining if the room dimensions are a one of a small room, a medium room, and a large room; and applying a standard dimension based on the determined room dimensions being the one of the small room, the medium room, and the large room.

In some aspects, the techniques described herein relate to a fragrance management apparatus, wherein the remaining scent is calculated based on a weight loss profile of the identified scent type and an operating time of the scent type in the scent container.

In some aspects, the techniques described herein relate to a fragrance management apparatus, wherein determining the removal rate further includes: determining an estimated run time of the remaining scent; and determining how long the remaining scent will last based on the weight loss profile and the estimated run time of the remaining scent.

In some aspects, the techniques described herein relate to a fragrance management apparatus, wherein the concentration level is an optimized intensity setting based on user preferences, room conditions, and environmental conditions.

In some aspects, the techniques described herein relate to a fragrance management apparatus, wherein causing the fragrance management apparatus to maintain the fragrance level based on the ppm output further includes: causing the fragrance management apparatus to operate one of a heater or a fan to cause a scent to be emitted from the fragrance management apparatus; determining a change on the operation of the one of the heater or the fan to maintain the fragrance level on the ppm output based on changes in one or more of the removal rate, the concentration levels, and environmental conditions; and adjusting the operation of the one of the heater or the fan to maintain the fragrance level on the ppm output.

In some aspects, the techniques described herein relate to a fragrance management apparatus, wherein the fragrance level is further based on user preferences.

In some aspects, the techniques described herein relate to a method of maintaining a fragrance level including: identifying a scent type in a scent container of a fragrance management apparatus; determining a room dimension where the fragrance management apparatus is installed; determining a removal rate of a remaining scent in the scent container; determining a concentration level based on settings of the scent container; calculating a ppm output of the fragrance management apparatus for the scent type based on the room dimension, the removal rate, and the concentration level; and causing the fragrance management apparatus to maintain a fragrance level based on the ppm output.

In some aspects, the techniques described herein relate to a method, wherein the scent type is a scent solution that emits a scent when heated and the scent container is a scent vial that contains the scent solution.

In some aspects, the techniques described herein relate to a method, wherein the room dimension is determined based on an identity of the room that categorizes the room dimensions.

In some aspects, the techniques described herein relate to a method, wherein categorizing the room dimensions further includes: determining if the room dimensions are a one of a small room, a medium room, and a large room; and applying a standard dimension based on the determined room dimensions being the one of the small room, the medium room, and the large room.

In some aspects, the techniques described herein relate to a method, wherein the remaining scent is calculated based on a weight loss profile of the identified scent type and an operating time of the scent type in the scent container.

In some aspects, the techniques described herein relate to a method, wherein determining the removal rate further includes: determining an estimated run time of the remaining scent; determining how long the remaining scent will last based on the weight loss profile and the estimated run time of the remaining scent.

In some aspects, the techniques described herein relate to a method, wherein the concentration level is an optimized intensity setting based on user preferences, room conditions, and environmental conditions.

In some aspects, the techniques described herein relate to a method, wherein causing the fragrance management apparatus to maintain the fragrance level based on the ppm output further includes: causing the fragrance management apparatus to operate one of a heater or a fan to cause a scent to be emitted from the fragrance management apparatus; determining a change on the operation of the one of the heater or the fan to maintain the fragrance level on the ppm output based on changes in one or more of the removal rate, the concentration levels, and environmental conditions; and adjusting the operation of the one of the heater or the fan to maintain the fragrance level on the ppm output.

In some aspects, the techniques described herein relate to a method, wherein the fragrance level is further based on user preferences.

In some aspects, the techniques described herein relate to a scent dispensing device that optimizes a scent output including: a non-volatile memory; and a controller of a scent dispensing device, the controller configured to execute instructions stored on the non-volatile memory to perform operations including: determining a desired scent intensity level setting based on user selected preferences; determining an identity of a scent in a scent container positioned within the scent dispensing device, the identity of the scent including a weight loss profile of the scent and a removal rate profile of the scent; determining a room dimension of a space where the scent dispensing device is positioned, the room dimensions being an estimated square footage of the space where the scent dispensing device is positioned; calculating a ppm output for the scent, the ppm output being an intensity level that the scent is output to fill the space with the scent to mimic the desired intensity level setting, the ppm output being calculated based on the weight loss profile of the scent, the removal rate profile of the scent, and the room dimension; and causing the scent dispensing device to emit the scent at the calculated ppm output.

In some aspects, the techniques described herein relate to a scent dispensing device, wherein causing the scent dispensing device to emit the scent further includes causing the scent dispensing device to control an operating level of one of a heater or a fan to disperse the scent out of the scent device based on the operating level of the one of the heater or the fan.

However, this list of features and advantages is not all-inclusive and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1 is a block diagram illustrating an example system for dynamic scent diffusion.

FIGS. 2A-2B illustrate example GUIs for gathering feedback and setting initial scent settings.

FIGS. 3A-3C depict examples of different room sizes for dynamic scent diffusion.

FIG. 4 depicts example GUIs for initial experience settings for dynamic scent diffusion.

FIG. 5 is a flowchart of an example method dynamic scent diffusion.

### DETAILED DESCRIPTION

The technology described in this disclosure relates to dynamically adjusting scent emission and/or intensity of a scent dispenser within a space. As an example, the technology allows for models of optimized scent dispensation to be developed and employed for automatic scent dispensation that is optimized for different spaces.

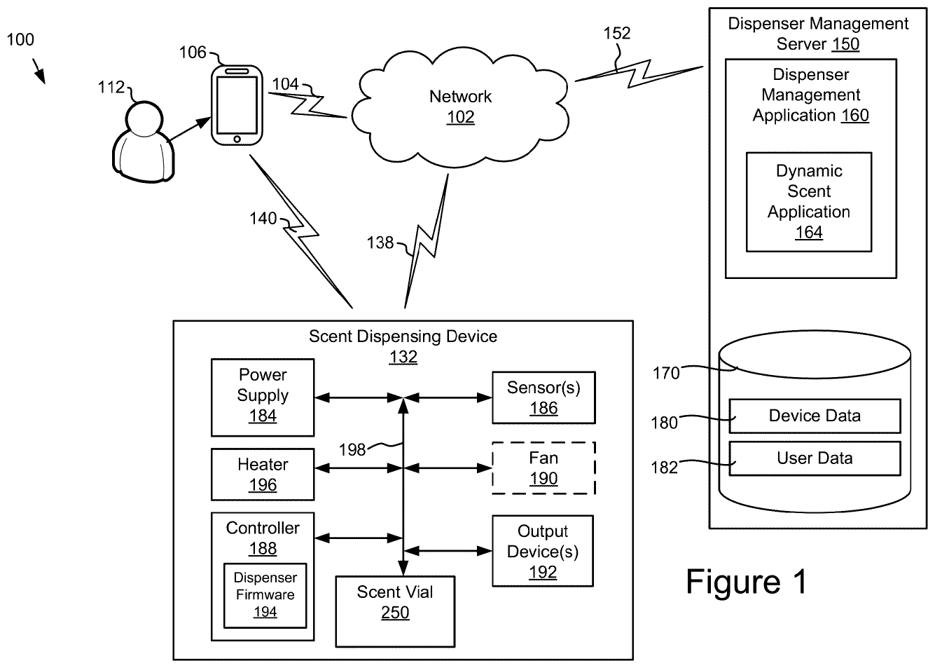

FIG. 1 is a block diagram illustrating an example system 100 for dynamically adjusting scent for scent dispensation. The system 100 may include one or more scent dispensing devices 132 positioned in an area, such as a room or space. In a typical implementation, a single scent dispensing device 132 is positioned within a space, however in large spaces multiple scent dispensing devices 132 may be positioned throughout the space for effective scent dispensing. In some implementations, the scent dispensing device 132 may plug into a wall outlet to provide power to the scent dispensing device 132. In other implementations, the scent dispensing device 132 may be battery powered, such as a car device or other portable device.

As shown, the illustrated system 100 further includes client device(s) 106 and a server 150, which are electronically communicatively coupled via a network 102 for interaction with one another and the scent dispenser(s) 132, etc., using standard networking protocols, as reflected by signal lines 104, 138, and 152. In some implementations, the scent dispensing device 132 may instead by communicatively coupled to the client device(s) 106, such as by Bluetooth, beacons, or other networking protocols (as reflected by signal line 140) in order to allow the scent dispensing device 132 to connect with a paired client device(s) 106 when in proximity rather than through the network 102.

The dispenser management application 160 operable by the dispenser management server 150 can receive operational data from the scent dispensing device 132 in association with the device 106 and/or user(s) 112 with which they are associated. The dispenser management application can receive management requests for a dispensing device 132, such as a diffusion level, scent choice, or various data that can be analyzed over time to improve the scent dispensing experience for the user 112.

The dispenser management application 160 can include a dynamic scent application 164. The dynamic scent application 164 may include software and/or logic for determining scent models and optimizing scent dispensation in one or more scent dispensing devices 132. The dynamic scent application 164 can provide setting information to the one or more scent dispensing devices 132 for dynamic adjustment of scent dispensation based on dynamic scent models.

The dispenser management server 150 includes a data store 170 storing various types of data used by the dispenser management application 160. Example data types include device data 180 and user data 182. The device data 180 may include a device model, a scent vial type, usage statistics, scent diffusion time, temperature variations, etc. The user data 182 may include entries for the users 112 of the system 100. A given entry may include a unique identifier for the user, a unique identifier for the user device 106, contact information for the user (e.g., address, phone number, electronic address (e.g., email)), payment information, scent subscription information specifying which reoccurring scent vials 250 should be shipped to the user, etc.

An example scent dispenser device 132 is depicted as including a power supply 184, one or more sensor(s) 186, a controller 188, output device(s) 192, dispenser firmware 194, a fan 190, a heater 196, and any number of scent vial(s) 250. The components 184, 186, 188, 190, 192, 196, and 250 are communicatively coupled via a communications bus 198. The controller 188 may include a non-transitory memory device (e.g., a non-volatile memory device), or may be coupled to a non-transitory memory device also coupled for communication via the bus 198. The non-transitory memory device may store software and/or firmware that specially configures the controller, such as the dispenser firmware 194. The power supply 184 may be any AC and/or DC power supply for powering the scent dispensing device 132. In some implementations, the power supply 184 may be battery powered and may be configured to charge when plugged into an AC and/or DC power supply or positioned on a wireless charging base. The controller 188 may be a microchip that controls the constituent electronics (e.g., sensor(s) 186, output device(s) 192, fan 190, heater 196, etc.) of the scent dispensing device 132.

The one or more sensor(s) 186 may include one or more temperature sensors for detecting the ambient temperature adjacent to the scent dispensing device 132 and can use those readings to determine efficient diffusion of the scent along with the dynamic scent settings from the dynamic scent application 164. In further implementations, the one or more temperature sensors may detect a temperature of one or more heating elements, such as of a heater 196 that may be used to emit a scent solution in a scent vial 250 by heating up the scent solution. In some implementations, the one or more sensor(s) 186 may include a vial sensor for sensing when to replace a scent vial(s) 250 installed in the scent dispensing device 132, optical or other sensor(s) or electronics for detecting an identity of scent vial(s) 250 installed in the scent dispensing device 132, ambient light sensor to detect a light level in a surrounding environment, and/or a motion sensor to detect motion in the surrounding environment, etc. In some embodiments, the sensors 186 may include a separate temperature sensor for the scent vial 250 (e.g., to measure temperatures at the scent vial 250, which can be used to adjust a speed and/or frequency of the fan 190).

The sensors 186 may include a transceiver having a wireless interface configured to communicate with the devices coupled to the network 102, such as the dispenser management server 150, and/or other components of the network 102 using standard communication protocols, such as Internet protocols. Further, the transceiver may be configured to wirelessly transmit data via a network to connect to other devices, such as the mobile device 106. By way of further example, the transceiver may transmit data to the mobile device 106 to which it is linked using a protocol compliant with IEEE 802.15, such as Zigbee®, Z-Wave®, Bluetooth®, or another suitable standard. Further embodiments are also possible and contemplated. In some embodiments, the transceiver may be embedded in the controller 188 or may be a component distinct from the controller and coupled to the controller 188 via the bus 198.

The output device(s) 192 may include light sources and/or audio reproduction devices, although further suitable output devices are also contemplated and applicable. In some implementations, the light sources and/or audio reproduction devices may be controlled to produce output consistent with a scent being emitted by the scent dispenser (e.g., a low, soothing light and music may be output in conjunction with a relaxing scent being emitted), or to communicate various alerts, such as low power, low scent vial levels, etc.

In some implementations, the scent dispensing device 132 may include the fan 190. The fan 190 may include a motor that has one or more fan blades that force air through the device 132 when the motor is operating. The fan 190 may operate at various speeds based on how quickly or slowly the motor runs. The fan 190 may be configured to nest within a housing of the device 132 and cause airflow to move through the device and across a scent vial 250 for scent diffusion or in alternative implementations, a scent tablet or gel may be used instead of a scent solution in a scent vial 250 for scent diffusion. In some implementations, the fan 190 may be able to turn on and off as signaled by the microcontroller 188 which results in substantially immediate scent diffusion. In some implementations the fan 190 and the motor speed may be associated with scent settings and based on the level of the motor operating the fan 190 different scent diffusions can be released to fill different areas over different times based on the scent settings.

The scent vial 250 may be removable and contain a liquid scenting agent that diffuses a scent into the nearby air. The scent vial 250 may be replaceable when the scent is diminished and a new scent vial 250 can be inserted into the device 132. The scent vials 250 may have various scent profiles and information about the scent vials 250 may be stored in the dispenser management application 160 to store the various scent profiles, ages of the scent vials 250, duration of use of the scent vials 250, exposed temperatures of the scent vials 250, etc.

The client device(s) 106 (also referred to individually and collectively as 106) are computing devices having data processing and communication capabilities. In some embodiments, a client device 106 may include a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The client devices 106 may couple to and communicate with one another and the other entities of the system 100 via the network 102 using a wireless and/or wired connection. Examples of client devices 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, car access panels, etc. In addition, while a single client device 106 is depicted in FIG. 1, it should be understood that any number of client devices 106 may be included.

In some implementations, the client device 106 may include a scent application, which allows the user to set scent dispensing device 132 settings, turn scent dispensers 132 on and off, purchase scent vials 250 for the scent dispensing device 132, provide feedback about optimized scent settings, set up a scent dispensing device 132, register an account, view analytics reflecting the user's historical use of his/her scent dispensing device 132, enable user profiles to use and setup scent profiles for the scent dispensing device 132, set a profile hierarchy (e.g., set which user profile(s) is/are the dominant user profile), set motor speeds for the fan 190, manage various scent settings, etc.

The dispenser management server 150 may include one or more computing devices having data processing, storing, and communication capabilities. For example, the server 150 may include one or more hardware servers, virtual servers, server arrays, storage devices and/or systems, etc., and/or may be centralized or distributed/cloud-based. In some embodiments, the server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the server 150 may include a (physical, virtual, etc.) processor, a non-transitory memory, a network interface, and a data store 170, which may be communicatively coupled by a communications bus. Similarly, the client device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 150 (e.g., the dispenser management application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 150 may include a dispenser management application 160 embodying a remotely accessible scent service. The dispenser management application 160 may send data to and receive data from the other entities of the system including the controllers 188, the mobile device(s) 106, etc. The dispenser management application 160 may be configured to store and retrieve data from one or more information sources, such as the data store 170. In addition, while a single server 150 is depicted in FIG. 1, it should be understood that one or more servers 150 may be included.

In some embodiments, the dispenser firmware 194, the scent application, the dispenser management application 160, the dynamic scent application 164, etc., may require users to be registered to access the acts and/or functionality provided by them. For example, to access various acts and/or functionality provided by the scent application, dispenser management application 160, and/or scent dispensers 132, these components may require a user to authenticate his/her identity (e.g., by confirming a valid electronic address). In some instances, these entities 132, 150, etc., may interact with a federated identity server (not shown) to register/authenticate users. Once registered, these entities 132, 150, etc., may require a user seeking access to authenticate by inputting credentials in an associated user interface.

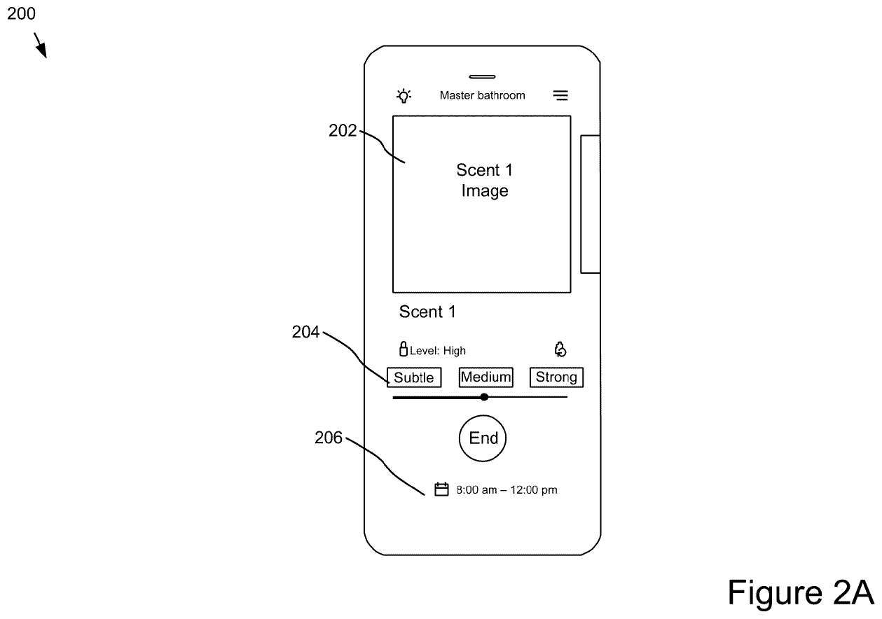

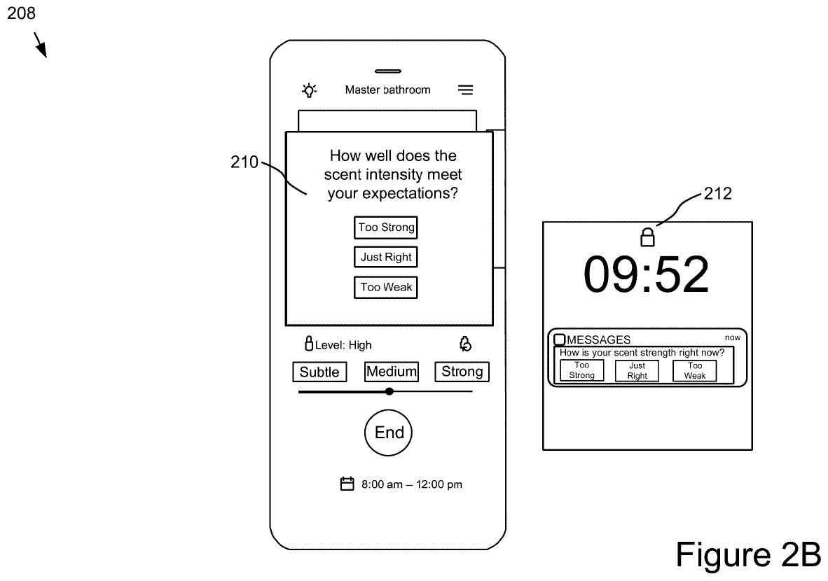

FIGS. 2A-2B depict graphical user interfaces of capturing data related to dynamic scent dispensation and/or configuring settings for dynamic scent dispensation. As shown in FIG. 2A, a computing device 106 may be used by a user 112 to select from a variety of different manual settings. For example, a graphical user interface (GUI) 200 as shown in FIG. 2A a user can select from a variety of scent intensity settings 204, such as a subtle setting, a medium setting, and/or a strong setting. In some implementations, a user may also be able to set one or more schedules 206 for a start/stop time of a setting or a duration of a setting. As shown in the GUI 200, a user 112 may select different settings for different scents 202. For example, one or more scents may be installed as scent vials 250 in the scent dispensing device 132 and a user 112 may select from the different scent vials 250 in the scent selection 202 of the GUI 200, as shown. As shown in FIG. 2A, a user may be able to manually configure a scent intensity level based on the intensity settings 204. In some implementations, those scent intensity settings may be configurable at various different levels, such as a slider that can allow for various scent intensity settings from low to high based on the slider settings, etc.

FIG. 2B depicts a GUI 208 for collecting feedback in optimized modeling for dynamic scenting. As shown in the GUI 208, users 112 may receive feedback options 210 and/or notifications 212 that allow them to provide opinions on the intensity settings and/or other settings of the scent dispensing device 132. The user 112 can provide feedback on intensity settings that the dynamic scent application 164 can use to generate dynamic scent models. The dynamic scent application 164 can then use the generated dynamic scent models for automatically adjusting scents for users 112 to optimize scent experiences without having the user 112 change or input manual settings. In some implementations, these generated dynamic models may take into account various scent data related to the scenting experience at the time the user provided feedback, such as a scent type, a scent remaining fill level, a scent temperature setting at the time of dispersion, a room size, a scent intensity setting, a user profile providing data related to user preferences, an ambient temperature, a detected air flow measurement, etc. The generated dynamic model can then adapt over time to determine optimal scent intensity settings for a user. For example, a user may want a “low intensity scent setting”, however, the dynamic scent application 164 may determine that in the current room location, with a currently measured temperature, and the scent profile of a current scent would render a low scent intensity setting to be undetectable to the specific user. In that example, the dynamic scent application 164 may then increase the scent intensity setting to mimic the users expected “low intensity” setting for the scent being emitted, while also allowing a higher level of scent emission to achieve the expected results for the user. These generated dynamic scent models can be further adjusted over time as additional feedback is provided and/or can use various machine learning algorithms to further optimize dynamic scent models based on similar scent profiles, etc. Based on the generated dynamic scent models, the dynamic scent application 164 can cause one or more of the scent dispensing devices 132 to operate at optimal experience temperatures for different experiences, such as subtle/medium/strong experiences, etc.

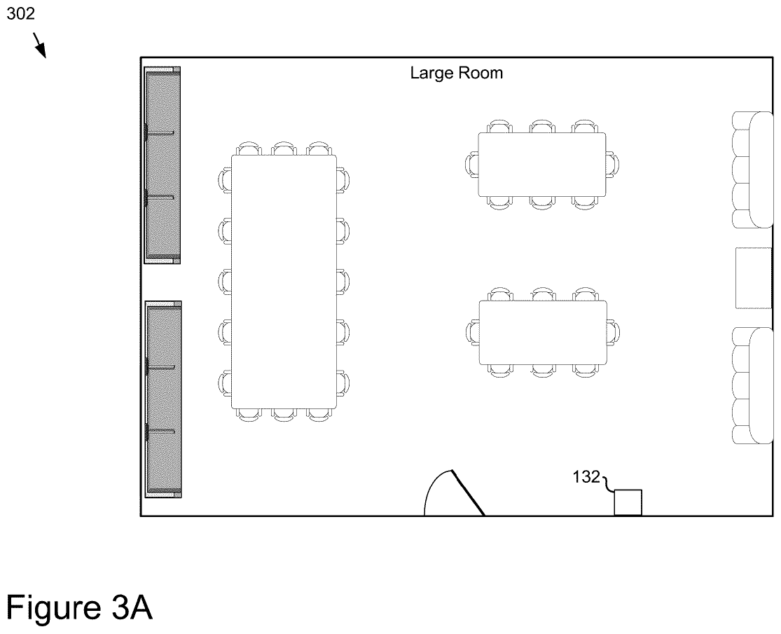

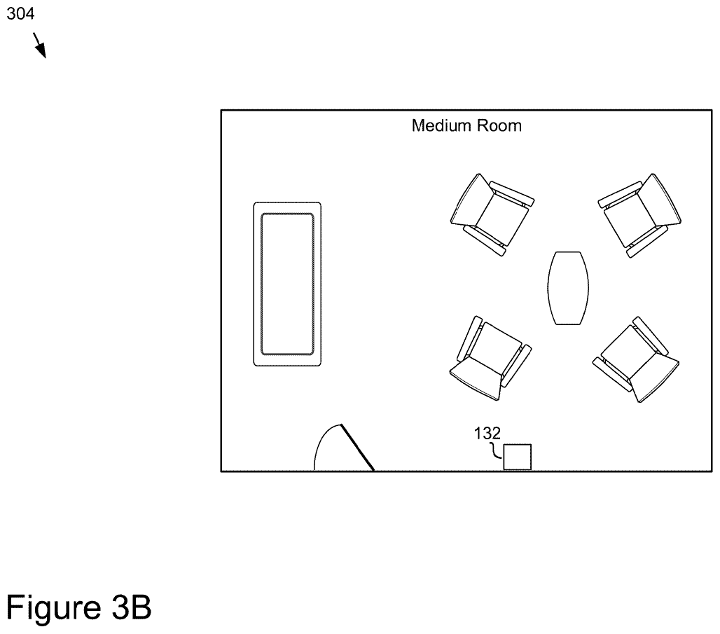

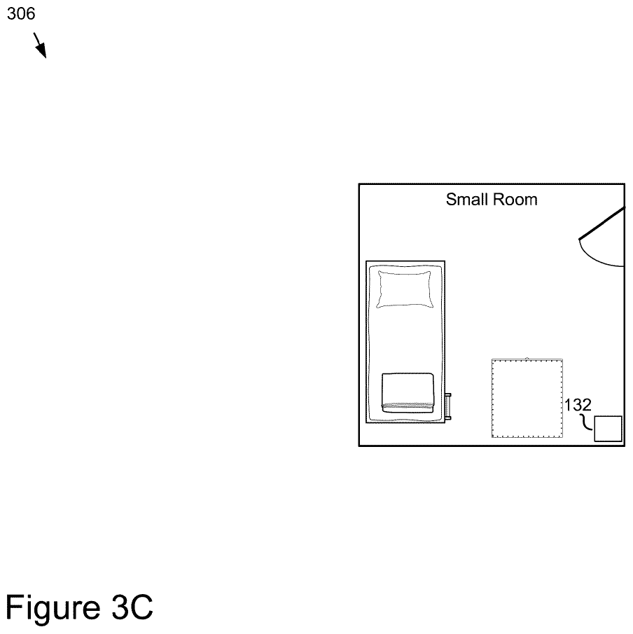

FIGS. 3A-3C depict different room sizes that a scent dispensing device 132 are installed in. Based on the different room sizes, the dynamic scent application 164 can adjust the intensity settings to further optimize scent dispensation in different sized rooms to optimize the various intensities in the different room sizes. The dynamic scent application 164 can integrate different variables, such as room size, desired scent strength or intensity, differences in scent sensitivity for users, environmental variables, surface area and/or materials in a room, etc. to optimize scent dispensation in different environments. For example, FIG. 3A depicts an example of a large room 302. In some implementations a large room may represent a basement, main bedroom, dining room, family room, guest room, living room, etc. such as in one example, with square footage greater than 180+ square feet. As shown in FIG. 3A, the scent dispensing device 132 may be positioned in the large room 302 and the dynamic scent application 164 can optimize the scent dispensation settings to disperse a desired scent level in the large room. In another example, FIG. 3B depicts an example of a medium room 304. In some implementations a medium room may represent a main bathroom, baby bedroom, guest bedroom, entryway, hallway, home office, kitchen, office, etc. such as in one example with square footage between 80-180 square feet. As shown in FIG. 3B, the scent dispensing device 132 may be positioned in the medium room 304 and the dynamic scent application 164 can optimize the scent dispensation settings to disperse a desired scent level in the medium room 304. In another example, FIG. 3C depicts an example of a small room 306. In some implementations a small room may represent a downstairs bathroom, upstairs bathroom, den, etc. such as in one example with square footage less than 80 square feet. As shown in FIG. 3C, the scent dispensing device 132 may be positioned in the small room 306 and the dynamic scent application 164 can optimize the scent dispensation settings to disperse a desired scent level in the small room 306.

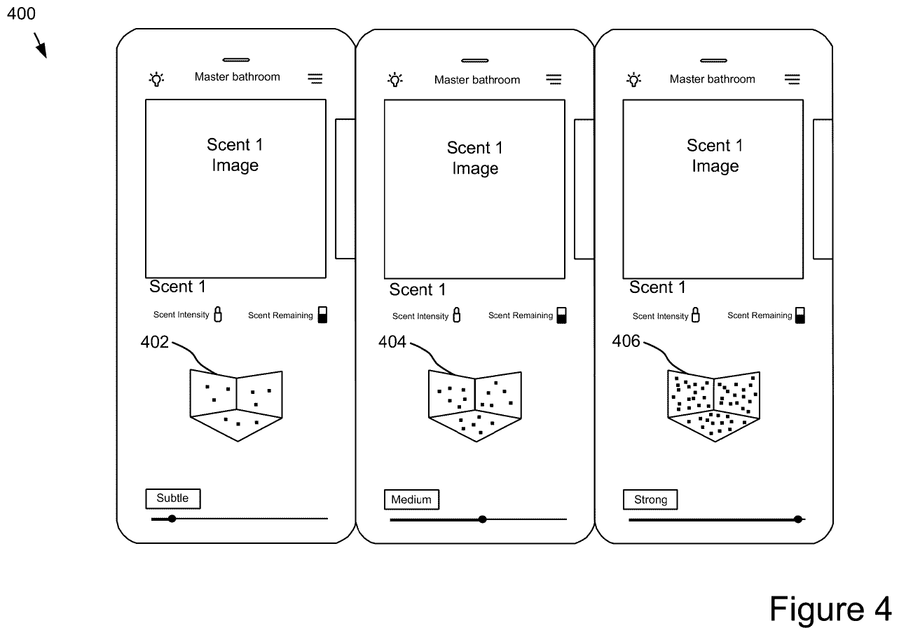

In some implementations, when a user 112 begins the initial setup experience to install a scent dispensing device 132, the dynamic scent application 164 can gather various information for use in generating/adjusting dynamic scent models. For example, this information can include power users, fragrance SKUs, room attributes, intensities, nightlight status, wearing time, times of day, geographic locations (such as shipping info and/or if away mode is active) customer service info, orders/month, number of devices, etc. This data can provide insights into the setup experience and further improve when users are having preferred experiences. As shown in FIG. 4, example GUIs depict different initial experience settings showing a graphical representation of a dispersion of scent molecules in a space, such as a subtle experience 402, a medium experience 404, and/or a strong experience 406.

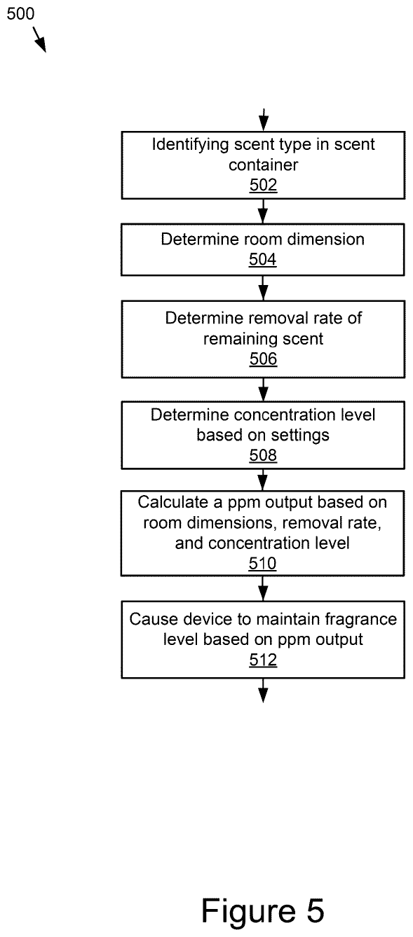

FIG. 5 is a flowchart of an example method 500 for optimizing a dynamic scent. At 502, the dynamic scent application 164 can identify a scent type in a scent vial 250. In some implementations, different scent types may have different scent outputs over time and/or may change their scent intensity based on the amount of scent solution remaining in a scent vial 250, etc. For example, a first scent may have a linear scent output as the scent is released over time, while a second scent may have a drop-off where the scent loses effectiveness over time. These scent models can be determined for different scent solutions and modeled for scent outputs over time. In some implementations, these scent models may be referred to as weight loss profiles of the scents or a removal rate profile of the scent. These profiles can be calculated by measuring the performances of specific scents in controlled environments and modeling how the scent changes over time and how the scent solution dissipates in the scent vial during operation.

At 504, the dynamic scent application 164 can determine a room dimension. In some implementations, the dynamic scent application 164 can determine room dimensions based on user inputs, such as room sizes/characteristics. In further implementations, the dynamic scent application 164 may receive location details, such as blueprints/plans and access locations information based on those location details. In some implementations, the dynamic scent application 164 may receive room dimensions from separate sensors to assist in determining the room dimensions and/or categorizing the room dimensions as small, medium, or large. At 506, the dynamic scent application 164 may determine a removal rate of the remaining scent. In some implementations, the removal rate of the remaining scent may be determined based on the scent models for different scent types. These scent models may be adjusted using various machine learning algorithms to optimize the scent models over time. In some implementations, the dynamic scent application 164 may determine a removal rate of the remaining scent based on weight loss profiles of various different scent models. In some implementations, these weight loss profiles may be determined using weight loss modeling to track weight losses, such as with a scale, percentage remaining, optical detection, concentration, etc. Tracking the weight losses of representative scent profiles, allows those scent profiles to be applied to the current scent remaining calculations. In other implementations, the removal rate of the remaining scent may be calculated for the specific amount of remaining scent in the scent vial 250. For example, a scale or other weight loss measuring device may be included in the scent dispensing device 132 and the scale or other weight loss measuring device may provided updated weights of the scent vial 250 as the scent solution is used. In further implementations, the remaining scent may be determined using optical sensors that detect a fill level of the scent over time as it is used in the scent vial 250.

At 508, the dynamic scent application 164 may determine a concentration level based on the settings, such as the settings for an optimized output for a specific user 112 in a specific room size. The concentration levels may be different for different scent types based on how scent types spread through different environments. The concentration levels may be measured as parts per million (“ppm”) of scent molecules in the space or environment. At 510, the dynamic scent application 164 can calculate a ppm output based on the room dimensions, the removal rate, and/or the concentration levels. At 512, the dynamic scent application 164 can send the ppm output to the scent dispensing device 132 and cause the device 132 to maintain a fragrance level based on the ppm output. In some implementations, the fragrance level may be an optimized and dynamically adjusted scent setting for particular users 112. In some implementations, maintaining a fragrance level, e.g., a scent diffusion level, based on the ppm can include maintaining or adjusting various heating or fan settings to cause the ppm output to change based on the scent profile or other factors. For example, if the scent profile shows a scent drop-off when only 25% of the scent solution remains, then when the dynamic scent application 164 determines that the remaining scent solution has dropped below the drop-off value, the dynamic scent application 164 may cause an internal scent intensity setting, such as an internal scent intensity setting not adjustable by a user, to be increased to account for the drop-off change. By allowing the dynamic scent application 164 to optimize and change various scent output settings to achieve a scent emission output that is optimized, the scenting experience is increased and a user is able to enjoy the scenting experience longer, such as when a scent vial is running out of scent solution or to maximize a scent emission experience over a period of time as the scent solution is being emitted.

In some implementations, the dynamic scent application 132 optimizes the scent experience by determining or modeling different variations of core wick temperatures of the scent container 250 that are needed to accomplish a desired ppm output for each different fragrance based on room size and with/without a nightlight based on a percentage of remaining scent in the scent container 250. The scent container 250 may have a wick that extends out of the container that can pull scent solution from the scent container 250 and be situated adjacent to a heater 196 or fan 190 to cause the scent to be emitted into the space from the heated wick. By optimizing the temperature of the core of the wick, the dynamic scent application 132 can control the variations in scent emission at different wick temperatures.

In some implementations, room size estimations may be done when the scent dispensing device 132 is initially installed in a room. For example, in some implementations, a user may be prompted to describe a room that the scent dispensing device 132 is initially being installed in, such as having the dynamic scent application 164 provide prompts to a user device 106 asking the user what type of room it is and categorizing the room based on common sizes for that type of room, or alternatively, the dynamic scent application 164 may prompt the user to provide estimated square footage of the room or space. In further implementations, the dynamic scent application 164 may receive a video stream or pictures from a user device 106 that show the layout of a room and using various AI algorithms, the dynamic scent application 164 can estimate a room size based on common sizes of items in the room, such as chairs, doorways, etc. The dynamic scent application 164 can further provide a guided scent installation experience where the dynamic scent application 164 determines a recommended location to install the scent dispensing device 132 and provides prompts to the user device 106 indicating where to install the scent dispensing device 132.

In one example implementation, the dynamic scent application 164 can first receive inputs such as a fragrance type (e.g., a scent solution identity), an intensity setting from the scent device 132, a fragrance composition through the life of the fragrance, and/or a nightlight setting (such as on or off, etc.). The dynamic scent application 164 may then be able to generate one or more predictive algorithms for how long the scent fragrance will last, such as when the scent solution will completely empty from a scent vial 250 or when a scent tablet or gel will lose all detectable scent as the fan 190 blows air over the scent tablet or gel. This algorithm for how long the scent fragrance may last is unique for each scent fragrance and or each users scent dispensing device 132 where the settings for intensity and nightlight operations may be different for various users. The dynamic scent application 164 may then also receive or continue to use various other inputs, such as the fragrance, the intensity, the fragrance composition, dissipation rate, diffusion rate, room size, nightlight setting, room conditions (such as lights, windows, fabrics, décor, air flow, etc.), nose sensitivity of a specific user, environmental conditions (such as ozone, hydroxide, humidity, temperature, etc.). Using those other additional inputs, the dynamic scent application 164 can then personalize a dynamic scent for a specific user and may generate a predictive algorithm for how long a scent related to the specific fragrance will be detectable in the room. The dynamic scent application 164 can then optimize various scent output settings based on a users desired scenting experience based on these predictive algorithms showing how long a scent would remain detectable at various different modeled settings from the predictive algorithms. By using the dynamic scent application 164 to create personalized scent experiences, users get a scent experience that is optimized to last longer, fill an appropriate amount of space, and maintain a desired intensity compared to when a user self-selects an intensity setting on a scent dispensing device 132.

In some implementations, where the scent solution is reactive to small changes in heat, the operation of one or more other components of the scent dispensing device 132 may change the output of the scent solution. For example, if a light, such as a nightlight is in operation when a scent is emitted, the heat emitting from the nightlight may affect the output of the scent solution. The dynamic scent application 164 can collect performance data showing how the scent changes when the nightlight or other components are operating and then factor those changes into the calculated output, such as to reduce a temperature of a heating element when it is determined that the nightlight is one, etc.

In some implementations, the scent dispensing device 132 may be configured to be portable and/or positioned within a vehicle. In this situation, where the scent dispenser 132 is moving and not located in a specific room, but instead may be in a transportable space, such as a car interior that can have various different environmental factors affecting the scent performance, such as high and low temperatures, changing air flow conditions, smaller sizes for scent emission space, localized scenting areas such as drivers area in a car instead of filling an entire room with scent, etc. The dynamic scent application 164 can factor in these additional changes to provide an optimal scent output for the smaller space such as the car. In further implementations, where the scent dispensing device 132 is portable, the scent dispensing device 132 could be positioned in an open space, such as outside. In these examples, the scent dispensing device 132 would not have the benefit of a closed area for scent emission. The dynamic scent application 164 can factor in that the scent dispensing device 132 is currently in an open space and can adjust the output of the scent to allow for an optimized scent emission for an open space.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A fragrance management apparatus, comprising:
a non-volatile memory; and
a controller of a fragrance management apparatus, the controller configured to execute instructions stored on the non-volatile memory to perform operations comprising:
identifying a scent type in a scent container of the fragrance management apparatus;
determining a room dimension where the fragrance management apparatus is installed;
determining a removal rate of a remaining scent in the scent container;
determining a concentration level based on settings of the scent container;
calculating a ppm output of the fragrance management apparatus for the scent type based on the room dimension, the removal rate, and the concentration level; and
causing the fragrance management apparatus to maintain a fragrance level based on the ppm output.

2. The fragrance management apparatus of claim 1, wherein the scent type is a scent solution that emits a scent when heated and the scent container is a scent vial that contains the scent solution.

3. The fragrance management apparatus of claim 1, wherein the room dimension is determined based on an identity of the room that categorizes the room dimensions.

4. The fragrance management apparatus of claim 3, wherein categorizing the room dimensions further comprises:
determining if the room dimensions are a one of a small room, a medium room, and a large room; and
applying a standard dimension based on the determined room dimensions being the one of the small room, the medium room, and the large room.

5. The fragrance management apparatus of claim 1, wherein the remaining scent is calculated based on a weight loss profile of the identified scent type and an operating time of the scent type in the scent container.

6. The fragrance management apparatus of claim 5, wherein determining the removal rate further comprises:
determining an estimated run time of the remaining scent; and
determining how long the remaining scent will last based on the weight loss profile and the estimated run time of the remaining scent.

7. The fragrance management apparatus of claim 1, wherein the concentration level is an optimized intensity setting based on user preferences, room conditions, and environmental conditions.

8. The fragrance management apparatus of claim 1, wherein causing the fragrance management apparatus to maintain the fragrance level based on the ppm output further comprises:
causing the fragrance management apparatus to operate one of a heater or a fan to cause a scent to be emitted from the fragrance management apparatus;
determining a change on the operation of the one of the heater or the fan to maintain the fragrance level on the ppm output based on changes in one or more of the removal rate, the concentration levels, and environmental conditions; and
adjusting the operation of the one of the heater or the fan to maintain the fragrance level on the ppm output.

9. The fragrance management apparatus of claim 1, wherein the fragrance level is further based on user preferences.

10. A method of maintaining a fragrance level comprising:
identifying a scent type in a scent container of a fragrance management apparatus;
determining a room dimension where the fragrance management apparatus is installed;
determining a removal rate of a remaining scent in the scent container;
determining a concentration level based on settings of the scent container;
calculating a ppm output of the fragrance management apparatus for the scent type based on the room dimension, the removal rate, and the concentration level; and
causing the fragrance management apparatus to maintain a fragrance level based on the ppm output.

11. The method of claim 10, wherein the scent type is a scent solution that emits a scent when heated and the scent container is a scent vial that contains the scent solution.

12. The method of claim 10, wherein the room dimension is determined based on an identity of the room that categorizes the room dimensions.

13. The method of claim 12, wherein categorizing the room dimensions further comprises:
determining if the room dimensions are a one of a small room, a medium room, and a large room; and
applying a standard dimension based on the determined room dimensions being the one of the small room, the medium room, and the large room.

14. The method of claim 10, wherein the remaining scent is calculated based on a weight loss profile of the identified scent type and an operating time of the scent type in the scent container.

15. The method of claim 14, wherein determining the removal rate further comprises:
determining an estimated run time of the remaining scent;
determining how long the remaining scent will last based on the weight loss profile and the estimated run time of the remaining scent.

16. The method of claim 10, wherein the concentration level is an optimized intensity setting based on user preferences, room conditions, and environmental conditions.

17. The method of claim 10, wherein causing the fragrance management apparatus to maintain the fragrance level based on the ppm output further comprises:
causing the fragrance management apparatus to operate one of a heater or a fan to cause a scent to be emitted from the fragrance management apparatus;
determining a change on the operation of the one of the heater or the fan to maintain the fragrance level on the ppm output based on changes in one or more of the removal rate, the concentration levels, and environmental conditions; and
adjusting the operation of the one of the heater or the fan to maintain the fragrance level on the ppm output.

18. The method of claim 10, wherein the fragrance level is further based on user preferences.

19. A scent dispensing device that optimizes a scent output comprising:
a non-volatile memory; and
a controller of a scent dispensing device, the controller configured to execute instructions stored on the non-volatile memory to perform operations comprising:
determining a desired scent intensity level setting based on user selected preferences;
determining an identity of a scent in a scent container positioned within the scent dispensing device, the identity of the scent including a weight loss profile of the scent and a removal rate profile of the scent;
determining a room dimension of a space where the scent dispensing device is positioned, the room dimensions being an estimated square footage of the space where the scent dispensing device is positioned;
calculating a ppm output for the scent, the ppm output being an intensity level that the scent is output to fill the space with the scent to mimic the desired intensity level setting, the ppm output being calculated based on the weight loss profile of the scent, the removal rate profile of the scent, and the room dimension; and
causing the scent dispensing device to emit the scent at the calculated ppm output.

20. The scent dispensing device of claim 19, wherein causing the scent dispensing device to emit the scent further comprises causing the scent dispensing device to control an operating level of one of a heater or a fan to disperse the scent out of the scent device based on the operating level of the one of the heater or the fan.
